(19) United States
(12) Patent Application Publication
(10) Pub. No.: US 2018/0014786 A1
(43) Pub. Date: Jan. 18, 2018

(54) MULTI-SPLINE, MULTI-ELECTRODE CATHETER AND METHOD OF USE FOR MAPPING OF INTERNAL ORGANS

(71) Applicant: Dragon Medical Development Limited, Sandyford (IE)

(72) Inventor: David Keane, Dublin (IE)

(21) Appl. No.: 15/651,730

(22) Filed: Jul. 17, 2017

Related U.S. Application Data
(60) Provisional application No. 62/362,634, filed on Jul. 15, 2016.

(51) Int. Cl. A61B 5/00; A61B 5/042; A61B 17/00

(52) U.S. Cl. CPC A61B 5/6858; A61B 5/0422; A61B 2562/0209; A61B 2017/00292

(57) ABSTRACT

A catheter for mapping the heart simultaneously accommodates a plurality of intravascular devices each having multiple electrode pairs carried on an exterior portion thereof. Each of the of intravascular devices is advancable and retractable relative to the distal end of the tubular catheter body independent of other vascular devices.

### FIELD OF THE INVENTION

The disclosure relates to cardiac mapping, and, more particularly to a catheter with multiple splines each having multiple electrodes which are freely positionable to allow for more accurate mapping.

### BACKGROUND

Cardiac catheter ablation is a minimally-invasive procedure used to remove or terminate a faulty electrical pathway from sections of the hearts of those who are prone to developing cardiac arrhythmias such as atrial fibrillation, atrial flutter, atrial tachycardia, and ventricular tachycardia. If not controlled, arrhythmias may increase the risk of major adverse events including death. The catheter ablation procedure can be classified by energy source: including radiofrequency ablation and cryoablation. Cardiac catheter ablation involves advancing several flexible catheters in through the patient's blood vessels towards the heart. Electrical impulses are then used to induce the arrhythmia and local heating or freezing is used to ablate (destroy) the abnormal tissue that is causing the arrhythmia.

Atrial fibrillation mapping and ablation have undergone significant advances since the advent of pulmonary vein isolation in 1999. The main emphasis of atrial fibrillation ablation is now on detection of non-pulmonary vein atrial triggers and drivers, e.g. rotors, of atrial fibrillation particularly in patients with persistent atrial fibrillation. Recent advances in mapping and ablation techniques of atrial fibrillation and of ventricular tachycardias require detailed mapping of endocardial and/or epicardial potentials. The endocardial surface is not spherical, but is 3-dimensionally complex and has ridges and recesses. As a result, the endocardial surface is not adequately mapped by conventional basket/arrays devices such as those disclosed in any of U.S. Pat. Nos. 4,699,147, 5,846,196, 5,848,972, 8,224,416, and 8,346,339 due to less than optimal contact achieved and inadequate density and distribution of multielectrode splines and electrodes.

The limitations of current cardiac mapping baskets and arrays devices, include, but are not limited to, inadequate number/density of splines, inadequate number/density of electrodes on each spline, and inadequate or non-uniformly distributed contact with the complex atrial endocardial contours, e.g. bunching up of some splines and excessive separation of other splines which are not equidistant once deployed. The expansion of current basket design devices typically results in simultaneous equal expansion of all splines including those which already have achieved contact with partial expansion, thereby resulting in a spheroidal symmetric basket in the non-spheroidal non-symmetric cardiac chamber.

Accordingly, need exists for an apparatus and technique which can provide more uniform density of contact for global mapping of a heart chamber as well as the option for higher density mapping in regions of interest by achieving a mapping catheter configuration within the targeted heart chamber that can be customized to the patient's individual anatomy and dimensions.

### SUMMARY

Disclosed is a cardiac mapping catheter which enables three-dimensional (3D) real-time mapping of electrograms and simultaneous mapping of the entire cardiac chamber. The catheter comprises a plurality of free-standing, independently expandable, high density splines containing electrodes and and/or 3D positioning sensors/emitters. The disclosed cardiac mapping catheter provides the ability to advance each spline individually until optimal contact is achieved without effecting deployment and contact already achieved in the other splines. Once the entire cardiac chamber has been mapped, the splines can then be freely adjusted to increase the density of splines and electrodes on one area, e.g. multiple splines can be simultaneously placed on any wall or region of interest. In one embodiment, the mapping catheter may comprise a distal tip configuration which enables one or more of the splines to be free and unconnected to the other splines. In another embodiment, the distal ends of plural splines are coupled to a distal connector tip while still enabling each spline to be advanced individually and expanded to different degrees independently. In another embodiment, the distal ends of plural splines are coupled to a distal expansile ring for epicardial application.

Disclosed is a mapping catheter comprising a common valved introducer, a common lumen, a common distal end port; and a plurality of pre-shaped multi-electrode splines introducible individually or in groups through a standard transseptal sheath.

According to one aspect of the disclosure, a catheter comprises a multiplicity of low-profile (small French size) multielectrode splines, with each spline having a low profile enabling a higher number of electrodes on each spline and each spline being separately and independently advancable or retractable from the other splines.

According to another aspect of the disclosure, a catheter comprises a multiplicity of low-profile (small French size) multielectrode splines being separately and independently advancable or retractable from the other splines and whose distal ends are freely positionable within the cardiac chamber.

According to still another aspect of the disclosure, a catheter comprises a multiplicity of low-profile (small French size) multielectrode splines being separately and independently advancable or retractable from the other splines and whose distal ends are attached to a common distal cap.

According to yet another aspect of the disclosure, the inclusion of 3-D positioning sensor/emitter on splines enables 3-dimensional electro-anatomical reconstruction of all maps and provides the option of incorporating such reconstruction into commercially available systems, such as Mediguide, and Navex and others.

According to another aspect of the disclosure, one or more of the splines may be implemented with a guidewire comprising an elongate tubular body having a core wire extending therethrough to a tip at a distal end thereof. The distal end of the guidewire further comprises multiple pairs of small surface area, closely spaced electrodes electrically couplable to a current source at the proximal end of the guidewire. The electrodes can sense electrical signals from the myocardium immediately adjacent to the electrodes, can pace the heart and can also perform pace-entrainment mapping in addition to activation mapping.

According to another aspect of the disclosure, a mapping catheter comprises: an elongate tubular body extending between proximal and distal ends thereof; and a plurality of intravascular devices simultaneously disposable within the elongate tubular body, each of the plurality of intravascular devices having a plurality of electrodes carried on an exterior portion thereof; wherein each of the plurality of intravascular devices is advancable and retractable relative to the distal end of the tubular body independent of others of the plurality of vascular devices. In one embodiment, each of the plurality of intravascular devices has a distal tip which is coupled to a common distal cap which is positionable beyond the distal end of the elongate tubular body.

According to yet another aspect of the disclosure, a mapping catheter comprises: an elongate tubular body extending between proximal and distal ends thereof; and a plurality of intravascular devices simultaneously disposable within the elongate tubular body, each of the plurality of intravascular devices having a distal region with a preshaped with a curve; wherein each of the plurality of intravascular devices is advancable and retractable relative to the distal end of the tubular body independent of others of the plurality of vascular devices. In one embodiment, less than all of the plurality of intravascular devices have a same predefined curvature.

According to another aspect of the disclosure, a method for mapping comprises: A) introducing into a space a plurality of intravascular devices through a common tubular body, each of the plurality of intravascular devices having a plurality of electrodes carried on an exterior portion thereof; and B) advancing one of the plurality of intravascular devices relative to a distal end of the tubular body independent of others of the plurality of vascular devices so that one of the plurality of electrodes is proximate a greatest extent of the space.

### DESCRIPTION OF THE DRAWINGS

The various features and advantages of the present invention may be more readily understood with reference to the following detailed description taken in conjunction with the accompanying drawings, wherein like reference numerals designate like structural elements, and in which:

FIG. 1A illustrates conceptually a side, cutaway partial view of the distal region of the mapping catheter in accordance with the disclosure, as taken 1A-1A of FIG. 1B;

FIG. 1B illustrates conceptually an end view of the multi-spline, multi-electrode mapping catheter of FIG. 1A;

FIG. 10 illustrates conceptually a perspective view of the distal end of the catheter of FIGS. 1A-B with multiple individual splines deployed in accordance with the disclosure;

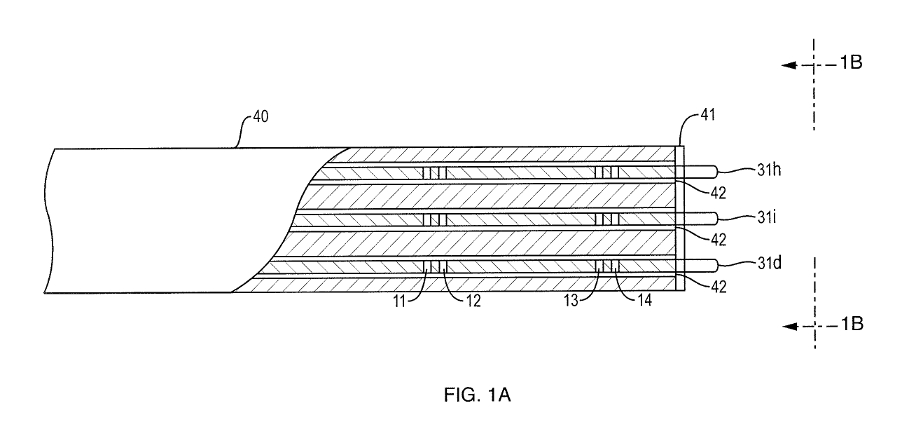

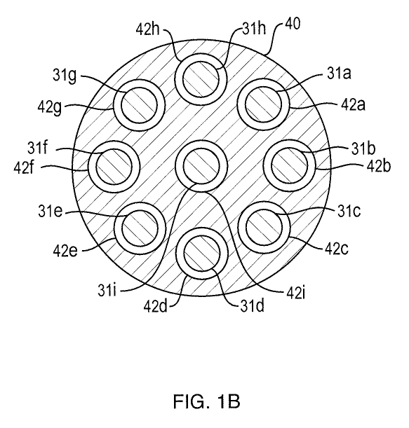

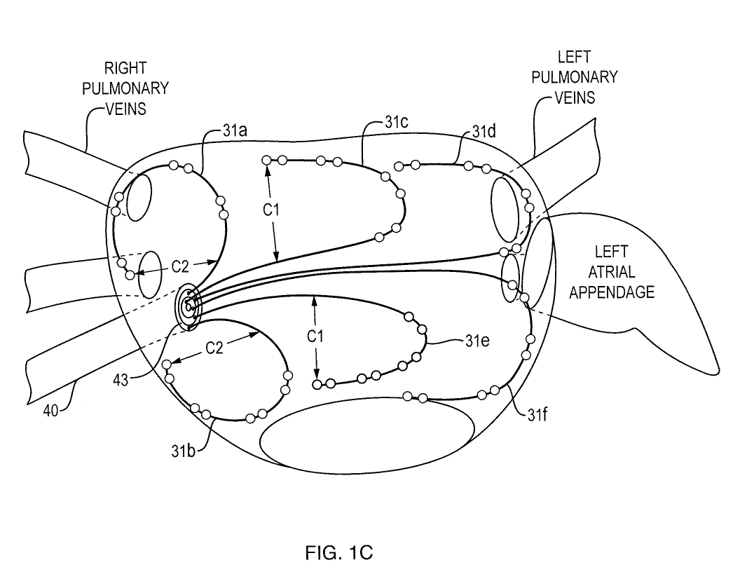

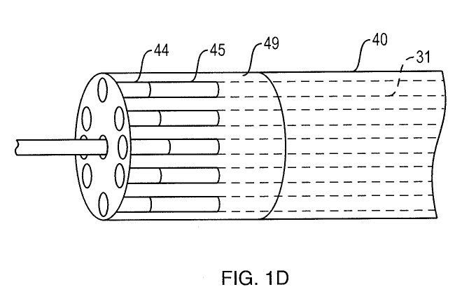

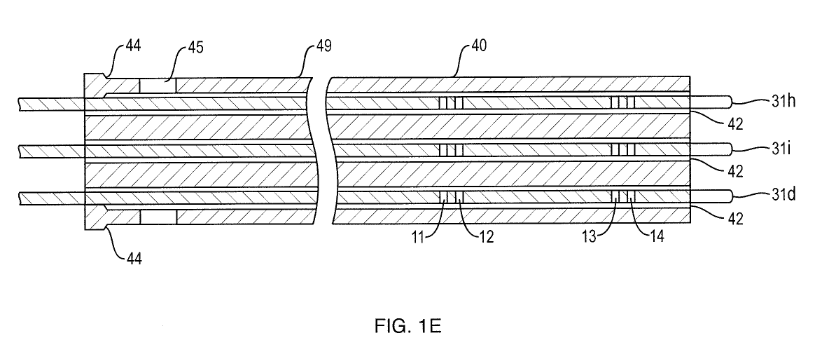

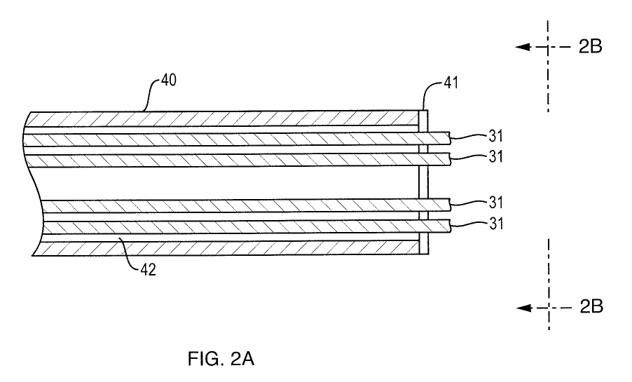

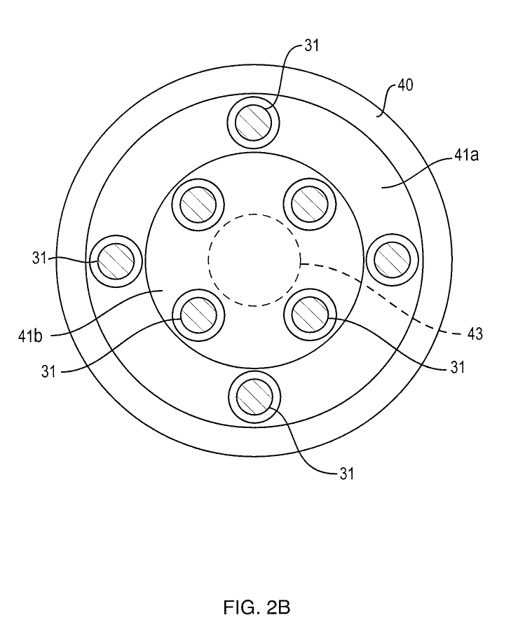

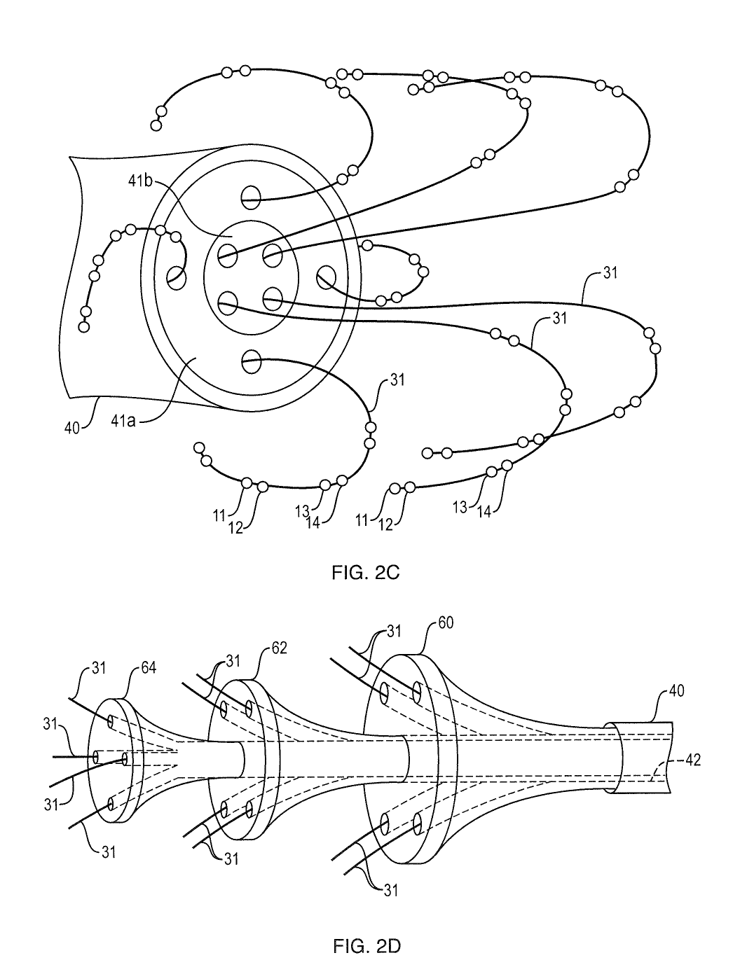

FIGS. 1D-E illustrate conceptually perspective and side, cutaway partial views, respectively, of the proximal actuation mechanism of the mapping catheter of FIGS. 1A-B;

FIG. 2A illustrates conceptually a side, cutaway partial view of the distal region of the mapping catheter in accordance with the disclosure, as taken 1A-1A of FIG. 1;

FIG. 2B illustrates conceptually an end view of the multi-spline, multi-electrode mapping catheter of FIG. 2A;

FIG. 2C illustrates conceptually a perspective view of the distal end of the catheter of FIGS. 2A-B with multiple individual splines deployed in accordance with the disclosure;

FIG. 2D illustrates conceptually perspective view of the proximal region of the mapping catheter of FIGS. 2A-B;

FIG. 3A illustrates conceptually a perspective view of an optional distal end cap for use with the catheters of FIGS. 2 and 3 in accordance with the disclosure;

FIG. 3B illustrates conceptually a perspective view of an optional stylus and distal end cap for use with the catheters of FIGS. 2 and 3 in accordance with the disclosure;

FIG. 4A illustrates conceptually a partial cutaway view of a spline guidewire in accordance with the disclosure; and

FIG. 4B illustrates conceptually a top view of a flat band wire suitable for use as a spline guidewire in accordance with the disclosure.

### DETAILED DESCRIPTION

The present disclosure will be more completely understood through the following description, which should be read in conjunction with the drawings. In this description, like numbers refer to similar elements within various embodiments of the present disclosure. The skilled artisan will readily appreciate that the methods, apparatus and systems described herein are merely exemplary and that variations can be made without departing from the spirit and scope of the disclosure.

FIGS. 1A-B illustrate conceptually a mapping catheter 30 in accordance with the disclosure. Mapping catheter 30 comprises, in an illustrative embodiment, an elongate tubular body 40 made of semi-rigid material, such as a natural or synthetic resin or polymers, having enough columnar strength to allow catheter 30 to be advanced into the cardiac chambers but flexible enough to negotiate curves within the blood vessels or arteries. Tubular body 40 has a plurality of interior lumens 42A-n extending therethrough from a proximal end to a distal end thereof, with each of the lumens 42A-n dimensioned to accommodate a spline 31A-n, respectively, slidably disposed therein, as illustrated. In the illustrated embodiment, without being limiting, mapping catheter 30 has ten interior lumens 42 to accommodate an equal number of splines 31, one of which may accommodate a standard guidewire to assist in advancement of the mapping catheter 30 into the atrial chambers. Other numbers of lumens 42 may be utilized as well, not all of which have the same dimensions or have to accommodate a spline 31. In one embodiment, all or a plurality of splines 31 may be implemented similar to guidewire 10, as illustrated in FIG. 3A. Other intravascular or intracardiac devices, such as balloon catheters, pressure sensing catheters, ablation catheters, all type of sheaths and guidewires, etc., may also be accommodated in an appropriately dimensioned lumen 42.

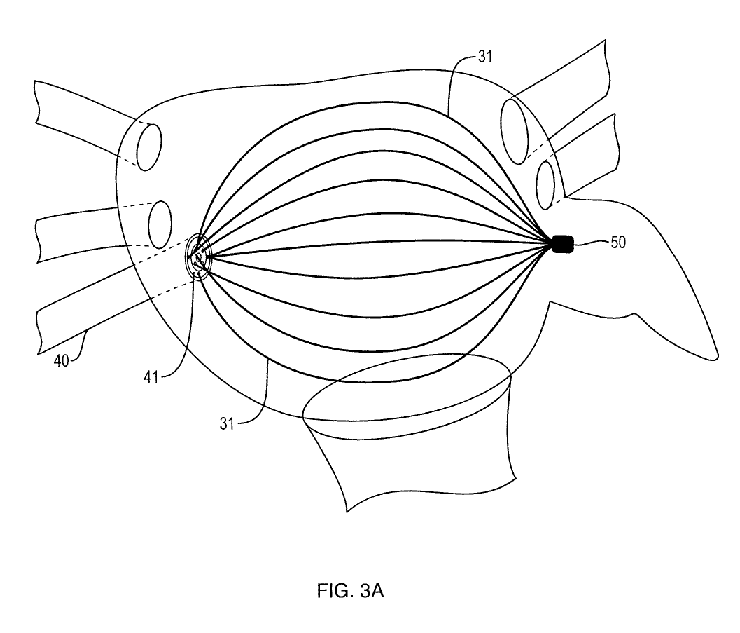

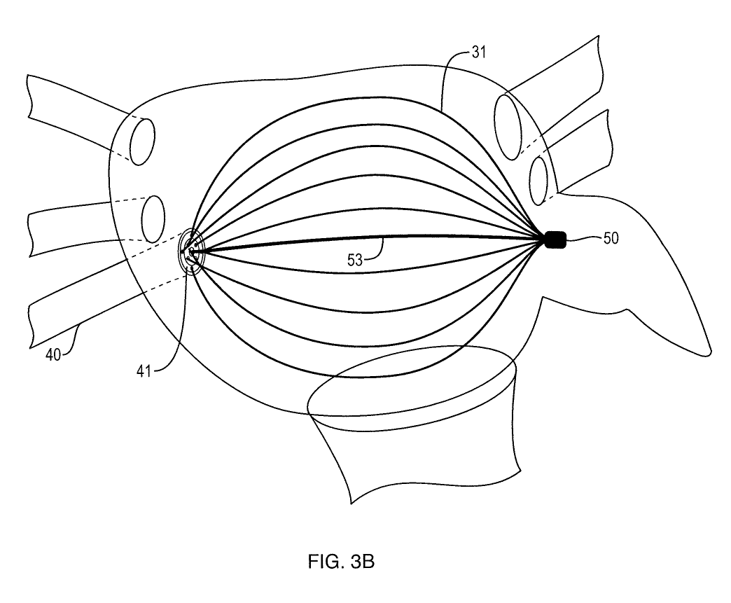

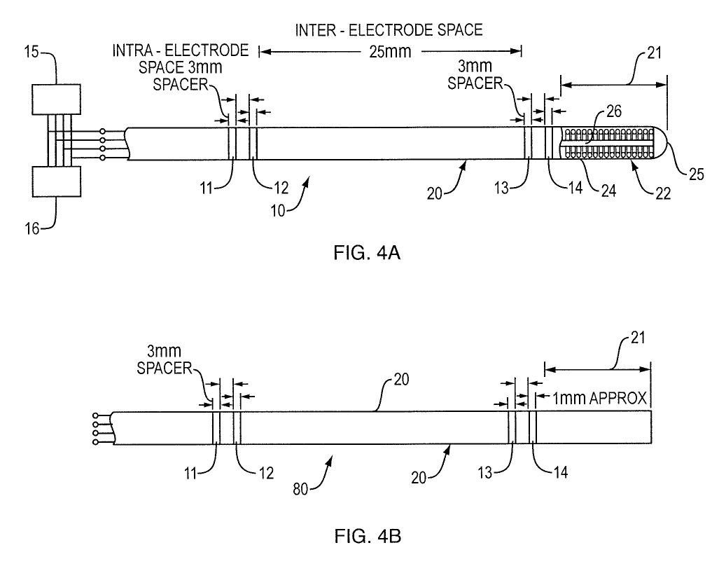

FIG. 1C illustrates conceptually a full chamber 3D mapping of electrograms in which transseptal delivery catheter 30 comprises, in one embodiment, multiple separate lumens and ports within the delivery sheath for each wire thereby enabling multielectrode splines to be advanced independently to achieve optimal contact for each spline individually and wide coverage of left atrium which is characterized by a contour for having multiple recesses and ridges in the atrial endocardium. As illustrated in FIG. 10, multi-electrode splines 31 may be pre-shaped to a predefined curvature C1 or C2, e.g. 180 degrees or 270 degrees, respectively. As illustrated in FIG. 10, splines 31 exit ports in the distal end port 41 of the distal end of catheter 30 into the left atrium of the heart. In embodiments, multielectrode splines 31 may have the same or different predetermined distal curve shapes, e.g. the first plurality of splines 31C-F may have a 180 degree curve for the left lateral aspect of the left atrium, and a second plurality of spines 31A-B may have a 270 degree curve for the right septal aspect of the left atrium. Other predetermined degrees of curvature may be in the range between 135 degrees to 315 degrees, or more specifically between 160 degrees to 290 degrees, or even more specifically between 180 degrees to 270 degrees. Note that the first and second plurality of splines 31 may have a prearranged grouping, e.g. all splines having a first curvature value arranged toward the center of the distal end port 41 or one side of the distal end port 41, or may be randomly arranged by the practitioner.

In embodiments, the wire implementation multielectrode spines 31 may have a lubricious hydrophyllic coating. The inner walls of lumens 42 may also have a lubricious hydrophyllic coating.

In the embodiment of FIGS. 1A-C, a plurality of individual small valved introducers, such as those disclosed in US Patent Application Publication US2005/0027257, may be in fluid communication with individual respective lumens 42 and the distal end at individual exit ports. The proximately located introducers may be all or partially comprised from or coated with a lubricious material, e.g. silicone. In embodiments, each spline 31 may emerge from the proximal end 47 of the main catheter lumen 42 through a manifold and into a separate tube having O-ring hemostasis seal.

In the embodiment of FIGS. 2A-C, a plurality of introducers, such as those disclosed in US Patent Application Publication US2005/0027257, may be combined with nested introducers 60, 62 and 64, and coupled to the proximal end 47 of the catheter 30 to be in fluid communication with common lumen 42 extending through the elongate tubular body 40.

In the embodiments, the axial position, and, therefore, the extent of each spline 31 protruding distantly from distal end port 43, may be implemented with an actuating mechanism coupled to the proximal handle 45, such actuating mechanism including a rotary control or thumb wheel mechanism mechanically coupled to one or more of splines 31 to allow advancement or retraction of the line relative to the distal end of catheter 30. Such an actuating mechanism is disclosed in US Patent Application Publication US201 seen in my ritual in FIG. 1 a no 7/0165064, such mechanism being able to advance and retract any of the splines 31 relative to the distal end of the tubular body independent of the other splines 31 or stylus 53.

In the embodiments of FIGS. 3A-B, the proximal handle 45 may implement individual slider controls 55 for each spline 31 attached to a distal cap 43, separate and apart from the mechanism to control the position of stylus 53, and, therefore, the position of distal cap 43.

FIGS. 1D-E illustrates conceptually a partial cross-sectional view of a proximal actuation mechanism 49 at the proximal end 47 of mapping catheter 30 containing individual slider controls 44A-n on actuation mechanism 49 to adjust each spine 31 approximately 2-3 cm. A plurality of O-rings may be used actuation mechanism to form a hemostasis seal in lumens 42A-n for each of spines 31A-n. The control 44 is manually actionable by depression against the spline 31 and the interior wall of lumen 42 enabling frictional engagement of the spline 31 and sliding control 44 in slot 45 in either direction along the axis of tubular body 40 to advance or retract the spline 31 accordingly.

In the embodiments of FIG. 2, the proximal actuation mechanism 49 may implement individual slider controls 44 for each spline 31 as well as a thumb wheel mechanisms to enable rotation of the distal end of a spline 31.

In the embodiments of FIGS. 3A-B, the actuation mechanism 49 may implement individual slider controls 55 for each spline 31 attached to a distal cap 43, separate and apart from the mechanism to control the position of stylus 53, and, therefore, the position of distal cap 43.

In embodiments, an optional gauge disposed at the proximal end 47 of catheter 30 can quantify length of a spline 31 advanced beyond the distal tip of tubular body 40 and may also quantify the amount of mechanical resistance, e.g. contact force, to further advancement of spline 31.

In the embodiments of FIGS. 1 and 2, although the individual electrodes splines 31 may be advanced and retracted over a range, e.g. 10 cm, within the target cardiac chamber being mapped, the splines 31 can also be completely withdrawn through the entire length of the mapping catheter and removed individually and/or collectively at the same time, in the event that individual splines exhibit evidence of mechanical kinking or demonstrate electrical noise or lack of electrical signal, enabling the problem spline to be removed and replaced individually.

In embodiments, multielectrode spines 31 and mapping catheter 40 may be coated with covalently-bonded-heparin as an anticoagulant. The mapping catheter 40 may have a proximal side arm, comprising a transparent material for bubble detection, for continuous flushing of heparinized saline fluid.

FIGS. 2A-B illustrate conceptually a mapping catheter 30, similar to that illustrated in FIGS. 1A-C, comprising an elongate tubular body 40, a distal end port 41 and a common lumen 42 extending therethrough from a proximal end to a distal end of elongate tubular body 40. Common lumen 42 is dimensioned to accommodate multiple spline 31A-n, slidably disposed therein, as illustrated. In the illustrated embodiment, without being limiting, mapping catheter 30 can accommodate eight splines 31. Other numbers of splines 31 may be utilized as well, not all of which have the same dimensions. In one embodiment, all or a plurality of splines 31 may be implemented similar to guidewire 10, as illustrated in FIG. 4. Other intravascular or intracardiac devices, such as balloon catheters, pressure sensing catheters, ablation catheters, all type of sheaths and guidewires, etc., may also be accommodated in an appropriately dimensioned lumen 42.

FIG. 2C illustrates conceptually mapping catheter 30 in which distal end port 41 has eight exit ports to accommodate eight spline 31A-n. Once in position, the multielectrode splines 31 with the greatest curvature are advanced distantly using the proximal control mechanisms described herein to exit through individual ports in the outer peripheral ring 41A of the distal end port 41 of catheter 30 so as to allow contact with and recordings made along the interatrial septum of the left atrium as well as the right side of the left atrium and the right pulmonary veins and antra. The multielectrode splines 31 with less curvature are advanced distantly using the proximal mechanisms described herein to exit through individual channels 42 in the inner peripheral ring 41B of the distal end port 41 of catheter 30 so as to allow contact with and recordings made along the main body of the left atrium and left lateral aspect of the left atrium and the left pulmonary veins and antra and the left atrial appendage.

In the embodiment of FIGS. 2A-C, a plurality of nested introducers 60, 62, and 64, each having four ports, as illustrated in FIG. 2D, are in fluid communication with common lumen 42 and maybe used to introduce up to twelve splines into lumen 42, with the individual introducing lumens illustrated phantom. Each of the ports in introducers 60, 62, and 64, may have its own O-ring hemostasis seal, or, alternatively, a single O-ring hemostasis seal paper provided at the point of coupling introducers 60 with the proximal end of elongate tubular body 40 and common lumen 42. Each of the ports in introducers 60, 62, and 64 may be further coupled to individual small valved introducers, or it may enable the practitioner to manually manipulate the proximal end of splines 31 directly therefrom.

FIGS. 3A-B illustrate conceptually embodiments of a mapping catheter 30 which may be substantially similar to the catheters of FIGS. 1 and 2 except that the distal ends of splines 31 are coupled to an optional distal tethering cap 50 which may be advanced or retracted relative to the distal end port of catheter 30 and which limits the actual amount that each individual spline 31 may be advanced beyond the distal end port 41.

In the embodiment of FIG. 3A, the distal tips of each of the multielectrode splines 31 may be attached to distal tethering cap 50 by compression or mechanical coupling, as illustrated, or in a manner similar to that disclosed in any of U.S. Pat. No. 8,224,416, US Patent Application Publication 20120271138, or with any other coupling technique. In such embodiment, distal tethering cap 50 is positioned as a result of the collective positions of the multielectrode spines 31. In this manner, using the proximal control mechanisms described herein each of the splines may bow outwardly individually to a different extent as the splines are each advanced independently so that their respective electrodes are proximate to the walls of the cardiac chamber, however, their respective ends are tethered longitudinally at the distal tethering cap 50. FIGS. 3A-B illustrate conceptually a full chamber 3D mapping of electrograms in which multielectrode splines 31 are attached to the distal tethering cap 50, while still enabling each of the multielectrode splines to be advanced independently to achieve optimal contact for each spline individually and wide coverage of left atrium which is characterized by a contour for having multiple recesses and ridges in the atrial endocardium.

In the embodiment of FIG. 3B, the distal tips of each of the splines 31 may also be attached to distal tethering cap 50, as illustrated, or in a manner similar to that disclosed in any of U.S. Pat. No. 8,224,416, Published, US Patent Application 20120271138, or with any other coupling technique. Distal tethering cap 50 is coupled to rod or stylus 53, in a manner reasonably understood by those skilled in the arts, which may be slidably disposed in a central lumen 42 of tubular body 40 and is positioned as a result of the extent to which stylus 53 extends distally beyond distal end port 41.

As with the embodiments of FIGS. 1 and 2, all or a plurality of splines 31 may be implemented similar to guidewire 10, as illustrated in FIG. 4. In the embodiments of FIGS. 3A-B, although the individual electrodes splines 31 may be advanced and retracted over a range, e.g. 8 cm, within the target cardiac chamber being mapped, the splines 31 cannot be completely removed and detached from the distal tethering cap 50 and continuously flushing lumen for fluid inside the catheter may be unnecessary.

In embodiments, the mapping catheter 30 may be manufactured as an assembly with splines 31 preloaded therein. A method for utilizing embodiments of mapping catheter 30 described herein is as follows. Catheter 30 may be introduced to the target heart chamber, e.g. into the left atrium, by transseptal access either through an outer deflectable or non-deflectable transseptal sheath or directly without an outer sheath achieved by an over-the-wire exchange from an initial transseptal sheath. With the over-the-wire exchange option, central lumen 42 of catheter 30, accommodates a delivery, e.g. transseptal, guidewire. In embodiments, mapping catheter 30 may be non-deflectable or deflectable by use of deflection wires embedded in the outer wall or inner core of the tubular body 40, in a manner understood in the art, to assist with proper placement.

Once in position, the multielectrode splines 31 with the greatest curvature are advanced distantly using the proximal control mechanisms described herein to exit through individual channels 42 in the outer peripheral ring of the distal end port 41 of catheter 30 so as to allow contact with and recordings made along the interatrial septum of the left atrium as well as the right side of the left atrium and the right pulmonary veins and antra. The multielectrode splines 31 with less curvature are advanced distantly using the proximal mechanisms described herein to exit through individual channels 42 in the inner peripheral ring of the distal end port 41 of catheter 40 so as to allow contact with and recordings made along the main body of the left atrium and left lateral aspect of the left atrium and the left pulmonary veins and antra and the left atrial appendage.

In embodiments, as part of the disclosed technique, an additional electrode may be added on the elongate tubular body 40, positionable in the inferior vena cava, to provide a reference for unipolar electrograms from the intracardiac spline electrodes 31.

In embodiments, as part of the disclosed technique, the degree of contact of each electrode (electrode pair) may be based on radiographic appearance, electrogram amplitude, electrogram high frequency components (which can be achieved by Fast Fourier Transform analysis and spectral analysis), electrogram fractionation, pacing threshold, impedance (myocardium has a higher impedance than blood) between each electrode and between neighboring electrode pairs, and by electrolocation whereby a charge applied between neighboring electrode pairs, e.g. the two electrodes of each pair being common, generates an electric field and changes in this field brought about by proximity to myocardium as opposed to the uniform conducting medium of blood is detected

FIG. 4A illustrates conceptually a partial cutaway view of a guidewire 10 that may be utilized as a spline 31. In an illustrative embodiment, the guidewire 10 may have design aspects similar to other commercially available PCI guidewires but with at least two pairs, and typically four pairs, of small surface area, closely spaced, electrodes disposed towards the distal end of the guidewire that can pace and sense the cardiac tissue immediately adjacent to the electrodes. These electrode pairs 11,12 and 13,14 are spaced apart axially along a length of the guidewire 10 in the more distal region thereof. The closer the intra-electrode distance, e.g., the distance between the two electrodes in a pair of electrodes, and the smaller the electrode size, the higher the resultant fidelity of the electrogram, and, the greater the desirable near field signal versus the undesirable far field signal. In embodiments, without being limiting, the individual electrodes may have a width of approximately 0.5 mm and an intra-electrode distance for a given pair of approximately 4 mm between individual electrodes within the same pair. In embodiments, without being limiting, the different pairs of electrodes may be separated, i.e. the interelectrode distance, by approximately 1 to 3 cm. Other spatial dimensions between electrodes in a pair, and between different pairs of electrodes may be utilized to optimize placement of the electrodes for the intended use. In embodiments, the ratio of inter-electrode spacing to intra-electrode spacing may be in the range between 5:1 to 40:1, or more specifically between 7:1 to 25:1, or even more specifically between 12:1 to 18:1. Each of the electrodes of electrode pairs 11,12 and 13,14 are coupled to electrically conductive leads which extend proximally through the guidewire 10 and are electrically couplable to signal source 15 and measurement circuit 16 which receive and process the signals from electrode pairs 11,12 and 13,14, as well as other electrode pairs on the same spline 31 to measure any of cardiac electrograms, pacing, and impedance. In an illustrative embodiment, at least one or multiple of the multielectrode splines 31 have multiple pairs electrodes, e.g. four pairs of electrodes each for a total of eight electrodes per spline 31.

The electrode pairs may be formed of any biocompatible electrically conductive material as can the electrical leads extending proximally along the axial length of the guidewire and connectable to a signal source and measurement circuit module 16. In one embodiment, the electrode pairs and their respective leads may be designed for MRI compatability, e.g. gold electrodes and copper wire leads or carbon and/or plastics conductive materials. In embodiments, the electrode leads may be embedded in the wall of elongate cylindrical tube 20 for mechanical and electrical isolation.

Guidewire 10 includes an elongate cylindrical tube 20 of semi-rigid material, such as a natural or synthetic resin, having enough columnar strength to allow it to be advanced through tortuous vasculature but flexible enough to negotiate curves within the vasculature. The guidewire exterior, particularly the distal end region 21 may have a polymer covered distal tip 22. The distal end of the guidewire 10 may be implemented with a helical platinum coil 24 coupled to a hemispherical bead tip 25, as illustrated in FIG. 4A. A core wire 26 extends through the interior length of the guidewire 10 and may have a cross-sectional diameter which narrows distally in either a stepped or progressively tapered manner. The core wire 26 may be coupled to one or both of the helical coil 24 or bead tip 25 to facilitate torqueing and steering of the guidewire 10. In one embodiment, the distal end region 21 of the guidewire 10 may be manually bendable or shapeable to retain a manually created curve, as in standard coronary guide wires. In another embodiment the distal tip 22 of the guidewire may be manually deflectable with an operator's handle from the proximal end of the guidewire in a manner understood from current commercially available steerable guide wires. In embodiments, the material from which the electrodes and/or guidewire tip are made may have increased radio-opacity for visual detection during placement of the guidewire. In embodiments, a portion of the guidewire, typically the distal tip region, may be made of a shape memory metal which reverts to a predetermined shape once reaching a threshold temperature within a patient vessel. In one embodiment, multielectrode splines 31 having preshaped distal ends may be larger in diameter and stiffer than a conventional floppy intra-coronary guidewire.

Proximal electrode pair 11,12 and distal electrode pair 13,14 may be carried on the exterior surface of cylindrical tube 20, either on the exterior diameter thereof or seated in indentations in the surface of tube 20, and are electrically coupled to leads which extend proximally through guidewire 10 for electrical coupling with the interface of measurement circuit module 16. Such electrical leads will be typically insulated and may extend through the hollow interior of 20 or may be embedded in the wall thereof. The source of a current signal may also be included within measurement circuit module 16. Note, in embodiments, the individual electrodes 11,12,13,14 may be coupled to a signal generator as well as measurement circuit module 16 in any configuration become as appropriate or in selectable configurations, e.g. electrode pairs electrically coupled in series with a signal generator or measurement circuit module 16, all electrodes in parallel with a signal generator or measurement circuit module 16, electrode pairs in parallel with a signal generator or measurement circuit module 16, or other configurations.

In practice, the guidewire 10 is connected to a signal source and measurement circuit module 16 and is inserted through a dedicated lumen or a common goal of catheter 30 and positioned within a cardiac chamber or other cavity inside the body.

In embodiments, one or more of the multi-electrode splines 31 can have an incorporated 3-D positioning sensor/emitter 52 located at the distal tip thereof to enable real-time electroanatomical mapping of the atrial chambers. In other embodiments two or three 3-D positioning sensor/emitter 52 may be located at reference sites along each spline 31 so that the precise location of the electrodes is known.

In embodiments, one or more of the multi-electrode splines 31 can have a soft distal end which is implemented with either a straight or J shaped coil. Such coil may be made or partially from a radiopaque material. Alternatively, a radio-opaque marker may be disposed at the distal end thereof. In other embodiments, the distal coil may be made of a shape memory material which assumes a predetermined shape once position in situ within the body.

The multielectrode splines 31 may be round, as illustrated in FIG. 4A, or may be flat tape/band shaped as illustrated in FIG. 4B. For the round wire multielectrode splines 31, the electrode pairs may be disposed on the outer curvature of the wire exterior, e.g. a 90 degrees to 180 degrees of arc of the wire diameter, of the shaped wire portion near the distal end thereof. Alternatively, the electrodes of each electrode pair may be ring or circumferential, e.g. 360 degrees of the diameter of the wire. Further, multielectrode splines 31 may be implemented with a band wire, having a non-round or rectangular cross-sectional shape, in which the electrodes may be are located on the outer surface of either side of the band wire portion in the distal region thereof. FIG. 4B illustrates conceptually a tape or band spline 80 with multiple electrode pairs similar to guidewire 10 except the splines and electrodes may have different shapes. Either of band wire or round wire splines 31 may be used with any of the embodiments described herein. A band wire 80 suitable for use with the disclosed mapping catheter is also described in US Patent Application Publications 20150223757 and 20140121470.

As indicated elsewhere herein although FIGS. 4A-B illustrate only two electrode pairs splines 31, whether implemented with guidewire 10 or band wire 80 may have at least four electrode pairs, for total of eight electrodes independently positional within a cardiac chamber using the devices and techniques disclosed herein. Further, the pairs of electrodes and splines 31 may be positioned substantially in the distal region of each splines 31.

Connections within or proximal (external) to the handle 45 the catheter 30 may be hardwired or may be via wireless communication, e.g. blue tooth/WiFi/medical band RF, non-contact communication to a recording-signal and processing-display system 15, 16. In embodiments, the handle 45 may also be implemented with a battery to allow pacing without a hard-wired external source. In embodiments, an external connection option comprises an optical fiber connection with simultaneous parallel multiple, e.g. 64, fibers or wires, or, alternatively comprises an optical fiber connection with pulsed sequential sampling from each of 64 electrodes.

The disclosed multi-electrode guidewire 10 may be combined with multiple similar guide wires into the catheter 30 to perform epicardial mapping as well as to deliver ablative radiofrequency or electric energy to cause deliberate ablation/cauterization of myocardium (sub-epicardium) directly or to cause deliberate occlusion of a coronary artery or coronary vein. Such epicardial application may be applied to the atrium or to the ventricle. Similarly our endocardial mapping catheter can be applied to the atrium or the ventricle.

It will be appreciated that any of the described aspects, features and options described in view of the disclosed methods apply equally to the system, measurement circuit module and guidewire device. It will be understood that any one or more of the described aspects, features and options as described herein can be combined. For purposes of clarity and a concise description, features are described herein as part of the same or separate embodiments, however, it will be appreciated that scope of the concepts may include embodiments having combinations of all or some of the features described herein.

It will be obvious to those reasonably skilled in the art that modifications to the apparatus and process disclosed here in may occur, including substitution of various components or values, without parting from the true spirit and scope of the disclosure.

What is claimed is:

1. A mapping catheter comprising:
an elongate tubular body extending between proximal and distal ends thereof; and
a plurality of intravascular devices simultaneously disposable within the elongate tubular body, each of the plurality of intravascular devices having a plurality of electrodes carried on an exterior portion thereof;
wherein each of the plurality of intravascular devices is advancable and retractable relative to the distal end of the tubular body independent of others of the plurality of vascular devices.

2. A mapping catheter comprising:
an elongate tubular body extending between proximal and distal ends thereof; and
a plurality of intravascular devices simultaneously disposable within the elongate tubular body, each of the plurality of intravascular devices having a distal region with a preshaped curve;
wherein each of the plurality of intravascular devices is advancable and retractable relative to the distal end of the tubular body independent of others of the plurality of vascular devices.

3. A mapping catheter according claim 1 wherein the plurality of intravascular devices are simultaneously disposable within a lumen extending between the proximal and distal ends of the tubular body.

4. A mapping catheter according to claim 1 wherein the plurality of intravascular devices are simultaneously disposable among a plurality of lumens extending between the proximal and distal ends of the tubular body.

5. A mapping catheter according to claim 1 wherein each of the plurality of intravascular devices is advancable and retractable independent of others of the plurality of vascular devices with the handle mechanism.

6. A mapping catheter according to claim 1 wherein each of the plurality of intravascular devices is advancable and retractable independent of others of the plurality of vascular devices with a handle mechanism coupled to the intravascular device proximate the proximal end of the tubular body.

7. A mapping catheter according to claim 1 wherein each of the plurality of intravascular devices has a distal tip which is coupled to a common distal cap, the distal cap disposed distal of the elongate tubular body distal end.

8. A mapping catheter according to claim 1 wherein at least one of the plurality of intravascular devices comprises an elongate shaft and a plurality of electrode pairs disposed on an exterior surface of the shaft.

9. A mapping catheter according to claim 1 in combination with a signal source and measurement circuit and capable of measuring any of cardiac electrograms, pacing, and impedance.

10. A mapping catheter according to claim 1 wherein the plurality of intravascular devices each has a distal region with a predefined curvature.

11. A mapping catheter according to claim 1 wherein at least one of the intravascular devices comprises a guidewire.

12. A mapping catheter according to claim 1 wherein at least one of the intravascular devices further comprises a three-dimensional positioning sensor or emitter disposed at the distal region thereof.

13. A mapping catheter according to claim 1 wherein at least one of the intravascular devices further comprises one of a J-shaped coil and C-shaped coil disposed at the distal region thereof.

14. A mapping catheter according to claim 1 wherein at least one of the intravascular devices further comprises a radio-opaque marker disposed at the distal region thereof.

15. A mapping catheter according to claim except claim 1 wherein each of the plurality of intravascular devices has a distal tip which is unattached and freely advancable beyond the distal end of the tubular body.

16. A mapping catheter comprising:
an elongate tubular body extending between proximal and distal ends thereof; and
a plurality of intravascular devices simultaneously disposable within the elongate tubular body, each of the plurality of intravascular devices having a distal tip which is coupled to a common distal cap, the distal cap positionable beyond the distal end of the elongate tubular body;
wherein each of the plurality of intravascular devices is advancable and retractable relative to the distal end of the tubular body independent of others of the plurality of vascular devices.

17. A mapping catheter comprising:
an elongate tubular body extending along an axis between proximal and distal ends thereof; and
a plurality of intravascular devices simultaneously disposable within the elongate tubular body, each of the plurality of intravascular devices having a distal region with a predefined curvature;
wherein each of the plurality of intravascular devices is advancable and retractable relative to the distal end of the tubular body independent of others of the plurality of vascular devices and
wherein less than all of the plurality of intravascular devices have a same predefined curvature.

18. A mapping catheter according to claim 17 wherein a first group of the plurality of intravascular devices have a predefined curvature having a first value and a second group of the plurality of intravascular devices have a predefined curvature having a second value different from the first value.

19. A mapping catheter according to claim 1 further comprising a first guide element containing a plurality of apertures for receiving and directing a first group of the plurality of intravascular devices at the distal end of the elongate tubular body.

20. A mapping catheter according to claim 1 further comprising a second guide element disposed at the proximal end of the elongate tubular body, the guide element containing a plurality of apertures for receiving and directing a first group of the plurality of intravascular devices towards the distal end of the elongate tubular body.

21. A mapping catheter according to claim 1 further comprising one or more valved introducers in fluid communication with a common lumen extending through the elongate tubular body.

22. A mapping catheter according to claim 21 in combination with an actuating mechanism disposed at the proximal end of the elongate tubular body, the actuating mechanism movably coupled to at least one of the plurality of intravascular devices to enable movement of the intravascular device relative to the elongate tubular body.

23. A mapping catheter according to claim 22 wherein the actuating mechanism enables rotational movement of the intravascular device relative to the central axis of the elongate tubular body.

24. A mapping catheter according to claim 17 wherein the predefined curvature of at least one of the plurality of intravascular devices is in the range between any of 135 degrees to 315 degrees, 160 degrees to 290 degrees, and 180 degrees to 270 degrees.

25. A method for mapping comprising:
A) introducing into a space a plurality of intravascular devices through a common tubular body, each of the plurality of intravascular devices having a plurality of electrodes carried on an exterior portion thereof; and
B) advancing one of the plurality of intravascular devices relative to a distal end of the tubular body independent of others of the plurality of vascular devices so that one of the plurality of electrodes is proximate a greatest extent of the space.

26. A method for mapping according to any of the claim 25 wherein at least one of the intravascular devices further comprises a three-dimensional positioning sensor or emitter disposed at the distal region thereof.

27. A method for mapping according to any of the claim 25 wherein at least one of the intravascular devices is coupled to one of a signal recording, signal processing or signal display device.

28. A method for mapping according to any of the claim 25 further comprising:
C) processing signals from the plurality of electrodes to map the defined space.
